(19) United States
(12) Patent Application Publication
(10) Pub. No.: US 2020/0179153 A1
(43) Pub. Date: Jun. 11, 2020

(54) ADJUSTABLE BACK SUPPORT APPARATUS

(71) Applicant: MENG-CHUN WANG, Taichung City (TW)

(72) Inventor: MENG-CHUN WANG, Taichung City (TW)

(21) Appl. No.: 16/698,112

(22) Filed: Nov. 27, 2019

(30) Foreign Application Priority Data
Dec. 7, 2018 (TW) 107144073

(51) Int. Cl. A61F 5/02

(52) U.S. Cl. CPC A61F 5/028; A61F 2005/0167

(57) ABSTRACT

A adjustable back support apparatus is provided, including: a back support, including a first joint portion, the first joint portion having an elongate slot and a fixation hole communicated with the elongate slot, the fixation hole including a first hole; a shoulder support, including a connection portion and a second joint portion connected with the connection portion, the second joint portion having through holes, the second joint portion movably inserted into the elongate slot so that one of the through holes of the second joint portion being movable to correspond to the fixation hole; an engaging member, swingably disposed on the first joint portion, the engaging member having a protrusive buckle, the protrusive buckle disengageably buckled within the first hole and one of the through holes, at least a part of the engaging member being movable between a blocking position and a release position.

### BACKGROUND OF THE INVENTION

### Field of the Invention

The present invention relates to an adjustable back support apparatus.

### Description of the Prior Art

People who have been suffering from lower back pains such as inflammatory back pain, intervertebral disc herniation, degenerative joint disease, usually need a support apparatus (such as a back support apparatus or a waist support apparatus) to support body and eliminate pain.

A conventional back support apparatus includes a back frame with a front opening corresponding to a shape of the vertebra, a Velcro brace-fastener connected with the back frame, and two connection portions respectively connected with the back frame. The back frame supports a vertebra of wearer's body, the Velcro brace-fastener encircles a brace of wearer's body and is positioned at the front side of the brace. Two ends of the Velcro brace-fastener are respectively connected with two opposite sides of the back frame. The two connection portions are respectively supported on two shoulders of the wearer's body. However, everyone's height is different, but the size of the connection portion of the conventional back support apparatus is fixed. As a result, a fixed-size of the connection portion does not suit any height of the wearers' body. It is inconvenient for using.

The present invention is, therefore, arisen to obviate or at least mitigate the above mentioned disadvantages.

### SUMMARY OF THE INVENTION

An object of the present invention is to provide an adjustable back support apparatus which provides quick adjustment.

To achieve the above and other objects, an adjustable back support apparatus is provided, including: a back support, including a first joint portion, the first joint portion having an elongate slot and a fixation hole communicated with the elongate slot, the fixation hole including a first hole; a shoulder support, including a connection portion and a second joint portion connected with the connection portion, the second joint portion having a plurality of through holes, the second joint portion movably inserted into the elongate slot so that one of the through holes of the second joint portion being movable to correspond to the fixation hole; an engaging member, swingably disposed on the first joint portion, the engaging member having a protrusive buckle, the protrusive buckle disengageably buckled within the first hole and one of the through holes, at least a part of the engaging member being movable between a blocking position and a release position; wherein when the engaging member is located in the blocking position, the protrusive buckle is disposed through the first hole, engaged within one of the plurality of through holes and within the elongate slot; when the engaging member is located in the release position, the protrusive buckle is disengaged from the through holes; wherein the first joint portion further includes a base plate and at least one restriction structure which is disposed on the base plate, and the base plate and the at least one restriction structure define the elongate slot.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a perspective view of an adjustable back support apparatus according to a first preferred embodiment of the present invention;

FIG. 2 is a breakdown drawing of the first preferable embodiment of the present invention;

FIG. 3 is another breakdown drawing of the first preferable embodiment of the present invention;

FIG. 4 is another breakdown drawing of the first preferable embodiment of the present invention;

FIGS. 5-6 are drawings showing the first preferable embodiment of the present invention in use;

FIG. 7 is a partial cross-sectional view of the first preferable embodiment of the present invention;

FIG. 8 is a partially-enlarged view of FIG. 7;

FIG. 9 is another partial cross-sectional view of the first preferable embodiment of the present invention;

FIG. 10 is a breakdown drawing of a second embodiment of the present invention.

### DETAILED DESCRIPTION OF THE PREFERRED EMBODIMENTS

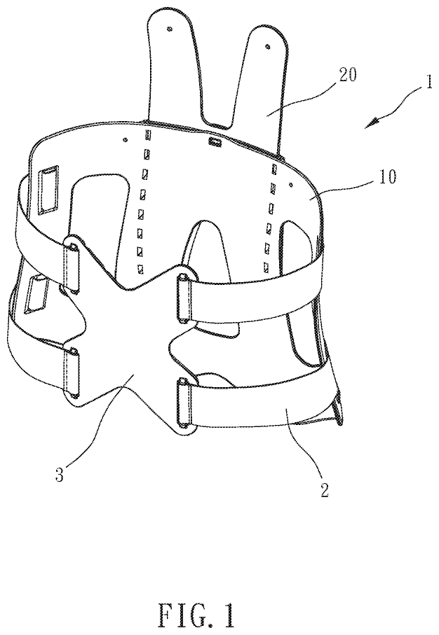

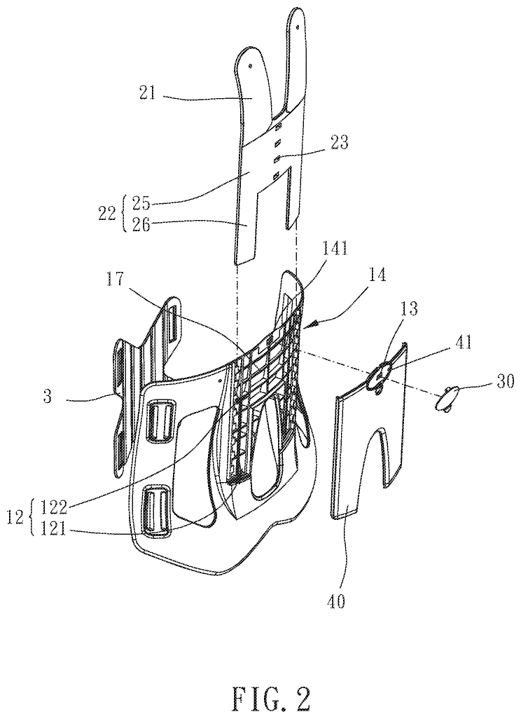

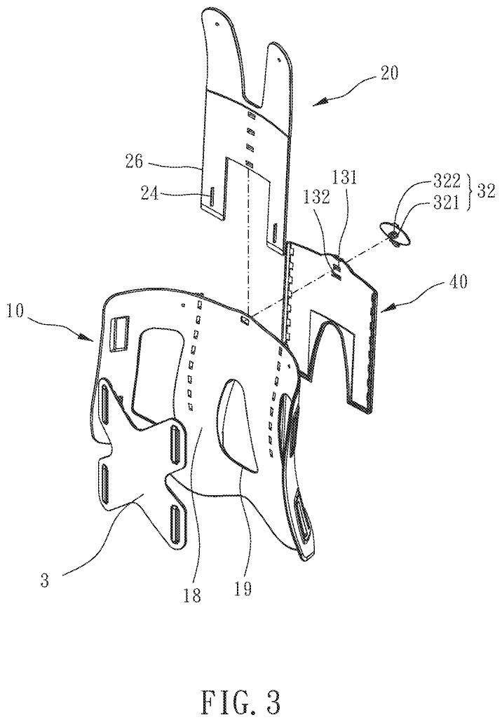

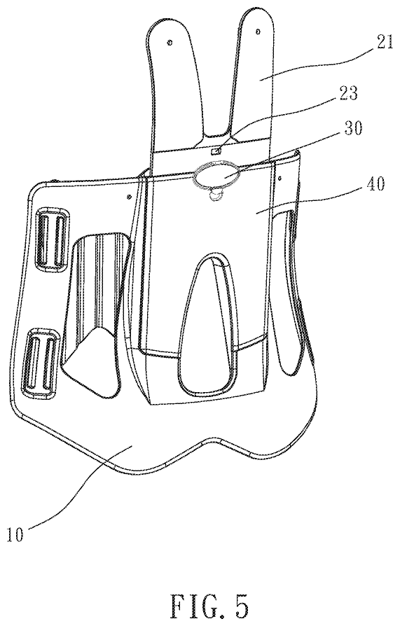

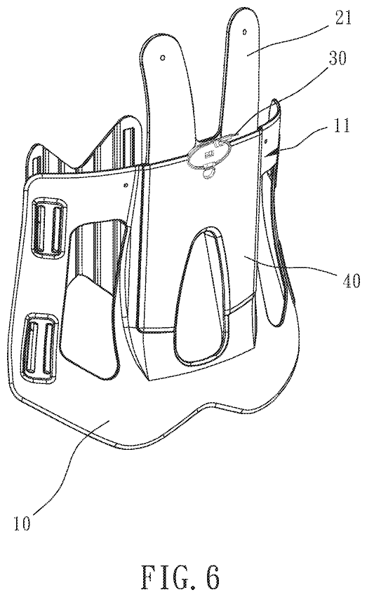

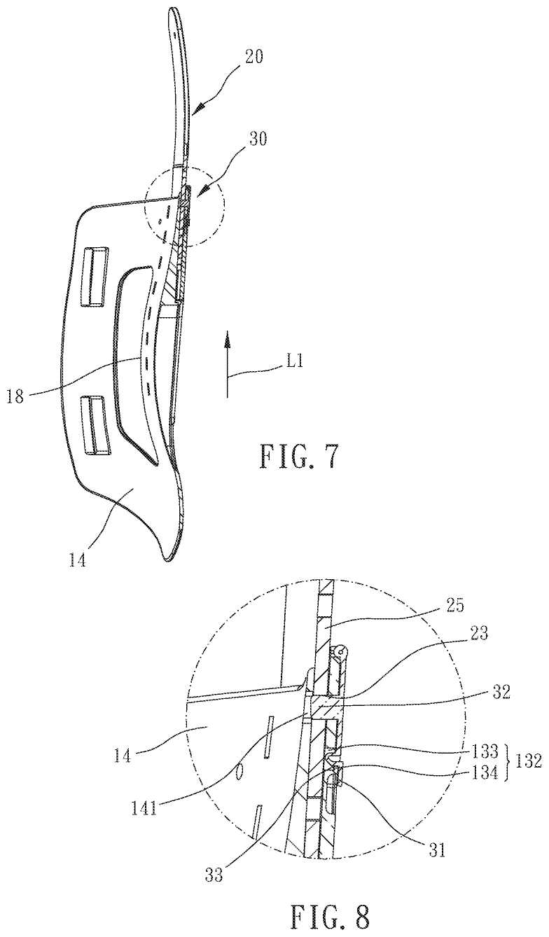

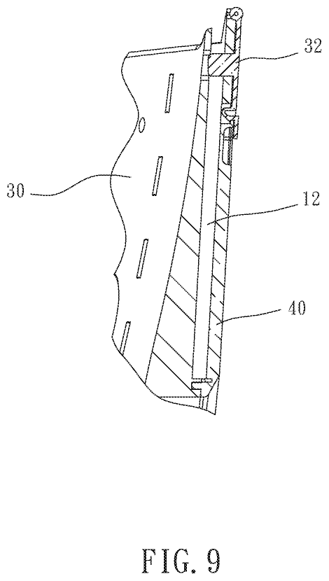

FIGS. 1-9 show an adjustable back support apparatus according to a preferred embodiment of the present invention. The adjustable back support apparatus 1 includes a back support 10, a shoulder support 20 and an engaging member 30.

Specifically, the back support 10 may be connected with a front support member 3 via a strap 2. The strap 2 may be an elastic strap. The front support member 3 and the back support 10 encircle a wearer's body, and respectively support the front of the wearer's body and the back of the wearer's body. The shoulder support 20 is configured to be supported the shoulder of the wearer's body. The shoulder support 20 is made of flexible material, which is easy to bend.

The back support 10 includes a first joint portion 11, the first joint portion 11 has an elongate slot 12 and a fixation hole 13 communicated with the elongate slot 12, the fixation hole 13 includes first hole 131. The shoulder support 20 includes a connection portion 21 and a second joint portion 22 connected with the connection portion 21, the connection portion 21 is supported the shoulder of the wearer's body or the back of the wearer's body. The connection portion 21 may be made of hard material or soft material. The second joint portion 22 has a plurality of through holes 23, the second joint portion 22 is movably inserted into the elongate slot 12 so that one of the through holes 23 of the second joint portion 22 is movable to correspond to the fixation hole 13. The engaging member 30 is swingably disposed on the first joint portion 11, and the engaging member 30 has a protrusive buckle 32. The protrusive buckle 32 is disengageably buckled within the first hole 131 and one of the through holes 23. At least a part of the engaging member 30 is movable between a blocking position and a release position. When the engaging member 30 is located in the blocking position, the protrusive buckle 32 is disposed through the first hole 131, engaged within one of the plurality of through holes 23 and within the elongate slot; when the engaging member 30 is located in the release position, the protrusive buckle 32 is disengaged from the through holes 23. In this embodiment, the engaging member 30 is pivotally disposed on the first joint portion 11, and may be integrally connected with the first join portion 11. The shoulder support 20 is movable relative to the back support 10, thus providing quick adjustment and stable positioning effect. Specifically, it can adjust the position between the shoulder support 20 and the wearer's body.

The first joint portion 11 further includes a base plate 14 and at least one restriction structure 15 which is disposed on the base plate 14, and the base plate 14 and the at least one restriction structure 15 define the elongate slot 12. In this embodiment, the elongate slot 12 opens toward a first direction L 1, the at least one restriction structure 15 includes at least one protrusion portion 16 protruding lateral to the first direction L 1; in other embodiment, the at least one restriction structure may be a cover which disengageably covers the elongate slot. Preferably, the first joint portion 11 includes two the restriction structures 15 which are respectively connected with two opposite sides of the base plate 14. Each of the two restriction structures 15 includes the at least one protrusion portion 16 toward one another, so as to prevent the shoulder support 20 from disengaging laterally from the long slot 12. In this embodiment, the two restriction structures 15 are disposed separately. Each of the two restriction structures 15 includes a plurality of the protrusion portions 16 disposed separately from each other. Each of the plurality of the protrusion portions 16 is a sheet-shaped member. As a result, it is easy to manufacture and is material-saving, and the shoulder support 20 can be stably movable relative to the back support 10. The base plate 14 has a buckle hole 141 which corresponds to the fixation hole 13, the engaging member 30 is engaged within the buckle hole 141, so as to reinforce combination.

The first joint portion 11 further includes the cover 40, the cover 40 disengageably covers the elongate slot 12. The engaging member 30 is disposed on the cover 40, the fixation hole 13 is disposed on the cover 40, the two restriction structures 15 located between the cover 40 and the base plate 14 so that it is easy to manufacture, assemble, and has strong structural strength. Specifically, the cover 40 is engaged within the elongate slot 12. Preferably, the cover 40 further has a receiving portion 41, the receiving portion 41 has the fixation hole 13. The engaging member 30 is received within the receiving portion 41, and thus the engaging member 30 can be restricted within the receiving portion 41 to prevent damage thereto when the engaging member 30 is swung. The engaging member 30 further has a pull portion 31 which is disposed separately from the cover so that it is easy to be pulled. In other embodiments, the cover and the base plate are integrally formed of one piece.

It is to be noted that, the base plate 14 has a plurality of ribs 17 which are respectively intersected with each other, so as to reinforce structural strength. In this embodiment, the plurality of ribs 17 are transverse to one another so as to reinforce the structural strength. In other embodiment, the ribs may be disposed in parallel.

The back support 10 further has a convex portion 18 configured to correspond to a lumbar vertebrae, the convex portion 18 corresponds a shape of the lumbar vertebrae, which is configured to perfectly fit and support the lumbar vertebrae. Preferably, the convex portion 18 has a via hole 19, which improves flexibility and prevents breaking. In this embodiment, the plurality of ribs 17 are respectively at least disposed around the via hole 19, so as to reinforce a structural strength.

The fixation hole 13 includes a holed structure 132, the holed structure 132 includes an inserting hole 133 and a hook 134 extending radially into the inserting hole 133, the engaging member 30 has a hook portion 3 which is disposed within the inserting hole 133 and disengageably engaged with the hook 134, and the protrusive buckle 32 is inserted into the first hole 131. In this embodiment, each of the first hole 131 and the inserting hole 133 is a through hole. In other embodiments, each of the first hole and the inserting hole 133 may be a blind hole. As viewed in a lateral direction, the hook portion 33 is a U-shaped hook and can be elastically deformed.

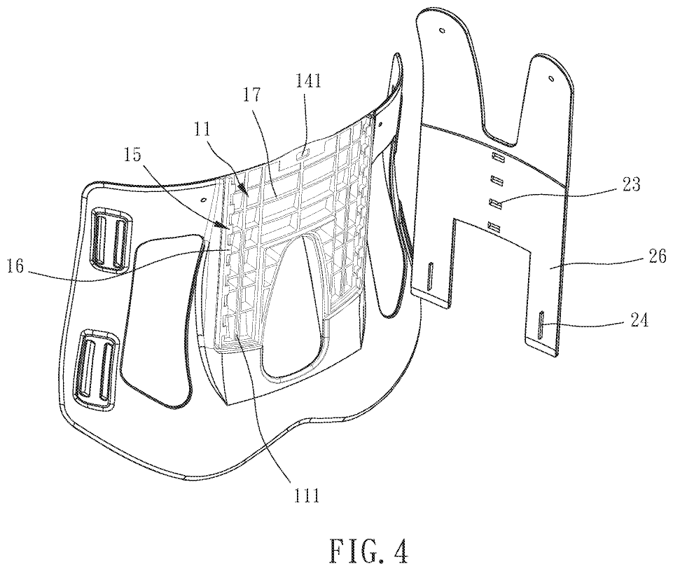

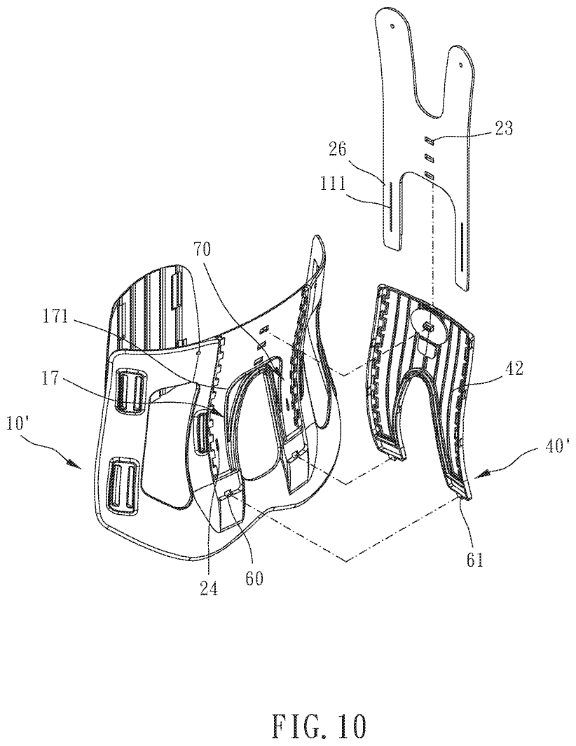

One of the first joint portion 11 and the second joint portion 22 has an elongated groove 111, the other of the first joint portion 11 and the second joint portion 22 has a bar 24 slidably engaged within the elongated groove 111. In this embodiment, as shown in FIG. 4, the first joint portion 11 has the elongated groove 111, and the second joint portion 22 has the bar 24. In other embodiment, as shown in FIG. 10, the arrangement of the bar 24 and the elongated slot 111 are opposite to the embodiment shown in FIG. 4. Respective numbers of the bar 24 and the elongated groove 111 are two. Each of the elongated grooves 111 and the bars 24 extends along the first direction L 1, so as to limit the traveling distance of the shoulder support 20. In other embodiments, respective number of the elongated slot and the bar may be less or more than two.

The protrusive buckle 32 is cross-shaped and abutted against a wall of the first hole 131. In this embodiment, the protrusive buckle 32 has an elongated convex section 321 and a short convex section 322, so as to prevent the protrusive buckle 32 from rotating relative to the first hole 131.

The shoulder support 20 further includes a body section 25 and two leg sections 26, the elongate slot 12 includes two narrowed section 121 and one widened section 122 communicated with the two narrowed sections 121. Each of the two leg sections 26 is inserted into one of the two narrowed sections 121, and the body section 25 is disposed within the widened section 122. Specifically, the shoulder support 20 is substantially H-shaped which is configured to be stably supported the shoulders and movable relative to the shoulders. In other embodiments, respective number of the leg section and the narrowed section can be less or more than two.

Please refer to FIG. 10 for a second preferred embodiment. In the second preferred embodiment, one of the cover 40′ and the back support 10′ has a plurality of inserting slots, the other of the cover 40′ and the back support 10′ has a plurality of inserting legs which are respectively inserted into one said inserting slots. In this embodiment, the cover 40′ has the plurality of inserting legs 61 and the back support 10′ has a plurality of inserting slots 60 for stable connection of the cover 40′ and back support 10′.

The back support 10′ has an arc-shaped section 70 which protrudes in a direction which is directed away from the cover 40′, the plurality of ribs 17 are disposed on the arc-shaped section 70, which reinforces structural strength. In this embodiment, each of the plurality of ribs 17 has a curved section transverse to the first direction L 1. The cover 40′ has a plurality of reinforced ribs 42, so as to reinforce structural strength.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. An adjustable back support apparatus, including:
a back support, including a first joint portion, the first joint portion having an elongate slot and a fixation hole communicated with the elongate slot, the fixation hole including a first hole;
a shoulder support, including a connection portion and a second joint portion connected with the connection portion, the second joint portion having a plurality of through holes, the second joint portion movably inserted into the elongate slot so that one of the through holes of the second joint portion being movable to correspond to the fixation hole;
an engaging member, swingably disposed on the first joint portion, the engaging member having a protrusive buckle, the protrusive buckle disengageably buckled within the first hole and one of the through holes, at least a part of the engaging member being movable between a blocking position and a release position;
wherein when the engaging member is located in the blocking position, the protrusive buckle is disposed through the first hole, engaged within one of the plurality of through holes and within the elongate slot; when the engaging member is located in the release position, the protrusive buckle is disengaged from the through holes;
wherein the first joint portion further includes a base plate and at least one restriction structure which is disposed on the base plate, and the base plate and the at least one restriction structure define the elongate slot.

2. The adjustable back support apparatus of claim 1, wherein the first joint portion includes two the restriction structures which are respectively connected with two opposite sides of the base plate.

3. The adjustable back support apparatus of claim 2, wherein the first joint portion further includes a cover, the cover disengageably covers the elongate slot, and the engaging member is disposed on the cover, the fixation hole is disposed on the cover, and the two restriction structures are located between the cover and the base plate.

4. The adjustable back support apparatus of claim 3, wherein the base plate has a buckle hole which corresponds to the fixation hole.

5. The adjustable back support apparatus of claim 2, wherein the elongate slot opens toward a first direction, each of the two restriction structures includes at least one protrusion portion protruding lateral to the first direction and toward one another.

6. The adjustable back support apparatus of claim 1, wherein the base plate has a plurality of ribs.

7. The adjustable back support apparatus of claim 1, wherein the back support further has a convex portion configured to correspond to a lumbar vertebrae.

8. The adjustable back support apparatus of claim 1, wherein the fixation hole further includes a holed structure which includes an inserting hole and a hook extending radially into the inserting hole, the engaging member has a hook portion which is disposed within the inserting hole and disengageably engaged with the hook, and the protrusive buckle is inserted into the first hole.

9. The adjustable back support apparatus of claim 1, wherein one of the first joint portion and the second joint portion has an elongated groove, and the other of the first joint portion and the second joint portion has a bar slidably engaged within the elongated groove.

10. The adjustable back support apparatus of claim 4, wherein the elongate slot opens toward a first direction, each of the two restriction structures includes at least one protrusion portion protruding lateral to the first direction and toward one another; each of the two restriction structures includes a plurality of the protrusion portions disposed separately from each other; the base plate has a plurality of ribs which are respectively intersected with each other; each of the plurality of the protrusion portions is a sheet-shaped member; the back support further has a convex portion configured to correspond to a lumbar vertebrae; the convex portion has a via hole, and the plurality of ribs are respectively at least disposed around the via hole; the fixation hole further includes the holed structure which includes an inserting hole and a hook extending radially into the inserting hole, and the engaging member has a hook portion which is disposed within the inserting hole and disengageably engaged with the hook, and the protrusive buckle is inserted into the first hole; the protrusive buckle is cross-shaped and abutted against a wall of the first hole; one of the first joint portion and the second joint portion has two elongated grooves, and the other of the first joint portion and the second joint portion has two bars which are respectively slidably engaged within one said elongated groove; the shoulder support further includes a body section and two leg sections, the elongate slot includes two narrowed sections and one widened section communicated with the two narrowed sections, each of the two leg sections is inserted into one of the two narrowed sections, and the body section is disposed within the widened section; the cover further has a receiving portion, the receiving portion has the fixation hole, and the engaging member is received within the receiving portion; the engaging member further has a pull portion which is disposed separately from the cover; one of the cover and the back support has a plurality of inserting slots, the other of the cover and the back support has a plurality of inserting legs which are respectively inserted into one said inserting slots; the back support has an arc-shaped section which protrudes in a direction which is directed away from the cover, the plurality of ribs are disposed on the arc-shaped section; the cover has a plurality of reinforced ribs.
